(19) United States
(12) Patent Application Publication
(10) Pub. No.: US 2023/0148636 A1
(43) Pub. Date: May 18, 2023

(54) FOOD COMPOSITION COMPRISING HEAT TREATED ALGAE

(71) Applicant: SOCIETE DES PRODUITS NESTLE S.A., Vevey (CH)

(72) Inventors: MICHAEL MERZ, Chicago, IL (US); VALERIE PATRICIA MAURICETTE MARIE AUDOUIN, Chicago, IL (US); INDIA C. SEVERIN, Lausanne (CH); MADLEEN SABOUNDJIAN, Lausanne 27 (CH); CHRISTOPH THOMAS WIDMER, Kehrsatz (CH); ROOSMARIJN FLEUR BULDER, GJ Amsterdam (NL)

(21) Appl. No.: 17/996,949

(22) PCT Filed: Apr. 26, 2021
(86) PCT No.: PCT/EP2021/060859

§ 371 Date: Oct. 24, 2022

(30) Foreign Application Priority Data
Apr. 27, 2020 (WO) 20171593.5
Apr. 27, 2020 (WO) 20171594.3
Apr. 27, 2020 (WO) 20171596.8
Apr. 27, 2020 (WO) 20171597.6

(51) Int. Cl. A23L 5/20; A23L 17/60; C12N 1/12

(52) U.S. Cl. CPC A23L 5/21; A23L 17/60; C12N 1/12; C12R 2001/89

(57) ABSTRACT

The present invention relates generally to the field of food compositions comprising algae. In particular, the present invention relates to a food composition comprising algae which are heat treated to reduce unwanted off-flavors. The algae may be algae from the Chlorella genus. For example, the algae may be Chlorella vulgaris. One embodiment of the present invention relates to a method to reduce the off-taste of an algae containing composition comprising a heat treatment, wherein the heat treatment comprises a primary heat treatment at a temperature in the range of about 130-160° C. and a secondary heat treatment at a temperature in the range of about 90-150° C.

The present invention relates generally to the field of food compositions comprising algae. In particular, the present invention relates to a food composition comprising algae which are heat treated to reduce unwanted off-flavors. The algae may be algae from the Chlorella genus. For example, the algae may be Chlorella vulgaris. One embodiment of the present invention relates to a method to reduce the off-taste of an algae containing composition comprising a heat treatment, wherein the heat treatment comprises a primary heat treatment at a temperature in the range of about 130-160° C. and a secondary heat treatment at a temperature in the range of about 90-150° C.

Algae are consumed by animals as feed and have also been used by humans as a part of their diet for a long time. Recent data published by the FAO estimate the global harvest of seaweeds in 2013 to have a value of about US $6.7 billion.

As a primary producer, algae convert light as an abiotic source of energy into organic compounds, which can in turn be used as an energy source by other organisms. As such, algae are by far the most abundant primary producers.

Algae are also an important source of vitamins, minerals, proteins, polyunsaturated fatty acids, or antioxidants for example. J Appl Phycol (2017) 29:949-982 reviews in this respect algae as nutritional and functional food sources.

Because algae represent an important source of micronutrients, antioxidants and natural colorants, for example, algae are increasingly used for the incorporation of these compounds into food and feed. For example, according to Food and Feed Research 40 (1), 21-31, 2013, by incorporating microalgae biomass in traditional food attractive and healthy new products may be designed.

For example, Chlorella, a genus of single-celled green algae belonging to the division Chlorophyta, may be considered as a potential source of food and energy.

One species of Chlorella is Chlorella vulgaris. Chlorella vulgaris has a long history of use as a food source and contains a unique and diverse composition of functional macro- and micro-nutrients. Curr Pharm Des. 2016; 22(2):164-73 summarizes the findings on the health benefits of Chlorella supplementation and the molecular mechanisms underlying these effects.

It is therefore not surprising that Chlorella vulgaris is often described as “superfood” and that there is an increasing interest of consumers in food compositions comprising Chlorella vulgaris.

However, unfortunately, Chlorella vulgaris preparations are well known to have an unpleasant off-taste. Because of this off-taste, many consumers do not enjoy Chlorella vulgaris containing food products as much as they would without the off-taste. One way to reduce the off-taste is to add lower amounts of algae to food products.

However, it would be desirable to have available algae preparations that can be used in foods or as food with less or no unpleasant off tastes.

Any reference to prior art documents in this specification is not to be considered an admission that such prior art is widely known or forms part of the common general knowledge in the field.

The objective of the present invention was it, hence, to improve the state of the art and in particular to provide a method to treat algae containing compositions to reduce and/or eliminate unwanted off-flavors so that they can be used in or as food compositions, or to at least provide a useful alternative.

The inventors were surprised to see that the objective of the present invention could be achieved by the subject matter of the independent claims. The dependent claims further develop the idea of the present invention.

Accordingly, the present invention provides a method to reduce the off-taste of an algae containing composition comprising a heat treatment, wherein the heat treatment comprises a primary heat treatment and a secondary heat treatment. The primary heat treatment may be carried out at a temperature in the range of about 130-160° C. The secondary heat treatment may be carried out at a temperature in the range of about 90-150° C.

The present invention further provides an algae containing composition obtainable and/or obtained by the method described in the present invention.

As used in this specification, the words “comprises”, “comprising”, and similar words, are not to be interpreted in an exclusive or exhaustive sense. In other words, they are intended to mean “including, but not limited to”.

The present inventors were surprised to see that if an algae containing composition, for example a composition comprising algae from the genus Chlorella, for example Chlorella vulgaris, is treated with a two-step heating process, unpleasant off-tastes that can be inherent to algae containing compositions can be reduced significantly or can even be removed completely.

Hence, the present invention relates to a method to reduce the off-taste of an algae containing composition comprising a heat treatment, wherein the heat treatment comprises a primary heat treatment and a secondary heat treatment. The primary heat treatment may be at a temperature in the range of about 130-160° C. The secondary heat treatment may be at a temperature in the range of about 90-150° C.

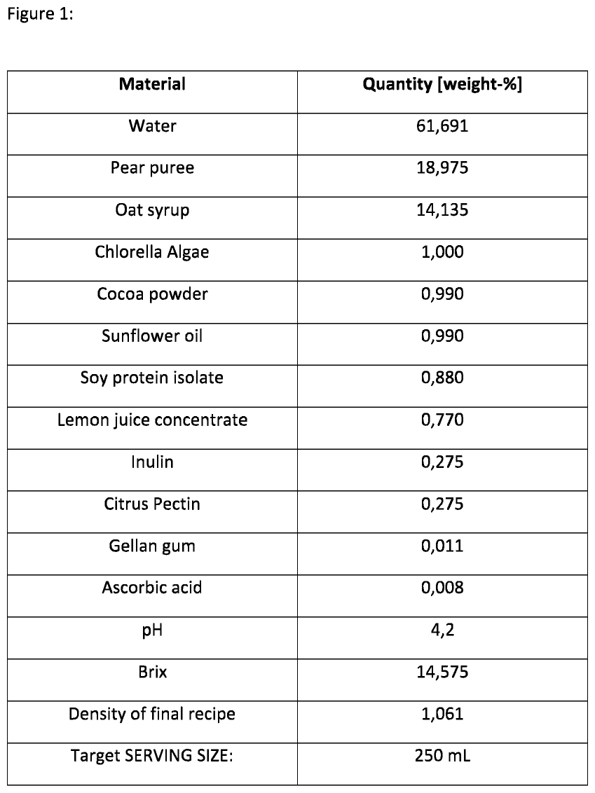

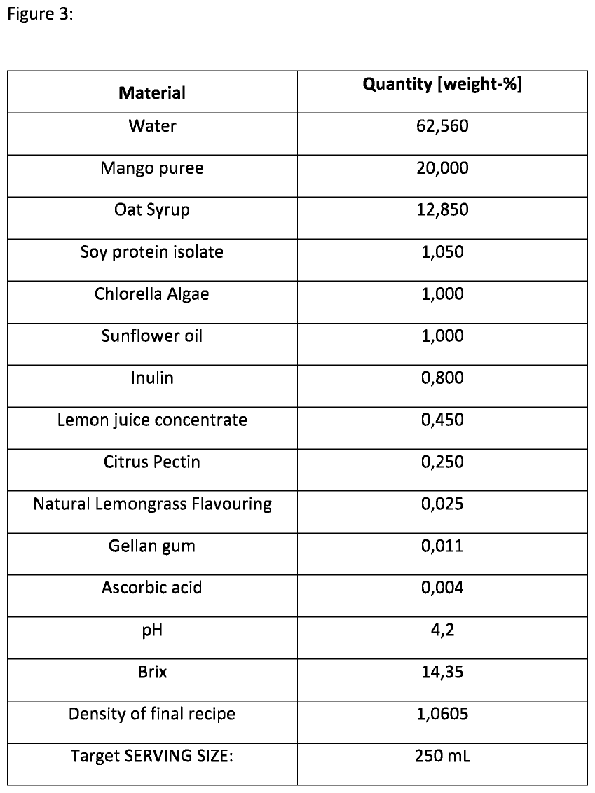

FIGS. 1-3 show recipes off typical liquid food compositions that the method of the present invention allows to prepare.

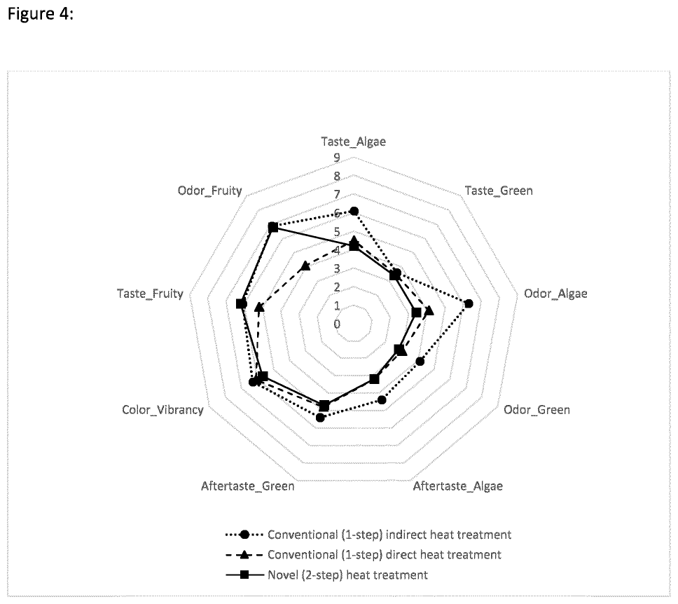

FIG. 4 shows the results of a sensory profiling of three algae drinks produced from the same recipe by applying different processes. All three drinks contain a total of 1% (w/w) of a mix of phototrophic and heterotrophic Chlorella. Conventional indirect heat treatment (dotted line), conventional direct heat treatment (dashed line) and the novel 2-step process (solid line) were compared in respect of the algae-like and green taste, odor and aftertaste as well as fruity taste and aroma.

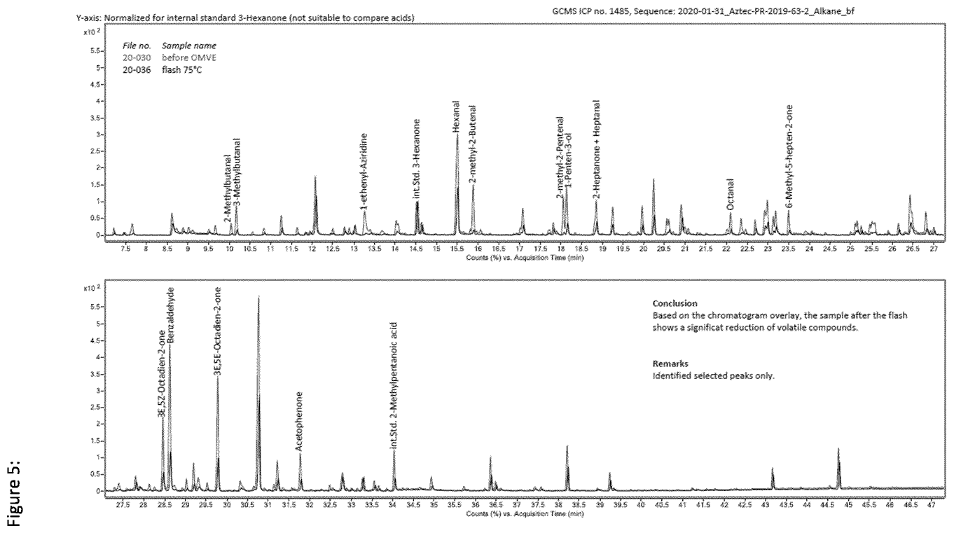

FIG. 5 shows the GC-MS chromatograms of the analysis of two algae solutions. Both algae solutions contain a total of 1% (w/w) of a mix of phototrophic and heterotrophic Chlorella. Samples were taken before and after direct heat treatment (145° C., for 5 s) followed by a flash cooling to 75° C.

Consequently, the present invention relates in part to a method to reduce the off-taste of an algae containing composition comprising a heat treatment, wherein the heat treatment comprises a primary heat treatment at a temperature in the range of about 130-160° C. and a secondary heat treatment at a temperature in the range of about 90-150° C.

The inventors have found that the heat treatments described in the present invention allowed it surprisingly to reduce and/or eliminate unwanted off-flavors in algae containing compositions.

This will be in particularly useful, if the algae containing composition is a food composition. Hence, for example, the algae containing composition may be a food product.

For the purpose of the present invention, the term “food” shall mean in accordance with Codex Alimentarius any substance, whether processed, semi-processed or raw, which is intended for human consumption, and includes drink, chewing gum and any substance which has been used in the manufacture, preparation or treatment of “food” but does not include cosmetics or tobacco or substances used only as drugs.

For example, the algae containing composition may be a liquid food composition, further, for example, a drinkable food product.

The term “liquid” food composition shall comprise all drinkable food compositions. The term “liquid” food composition may also comprise spoonable food compositions, for example, such food compositions that can be consumed without chewing.

The algae may be selected from the genus Chlorella. For example, the algae may be Chlorella vulgaris. The method of the present invention allowed it to achieve very good with Chlorella vulgaris.

Chlorella vulgaris algae is a green microalgae in the genus Chlorella. Chlorella vulgaris has a relatively high protein content, and—for example for this reason—has been used as food source, in particular in Asia, but increasingly also in other parts of the world.

Chlorella vulgaris is commercially available from many suppliers as food ingredient. For example, Chlorella vulgaris may be obtained from Allmicroalgae. For certain applications it may be preferred if the Chlorella vulgaris used for the present invention is certified as organic.

The inventors were surprised to see, that the unpleasant algae off-tastes can be particularly well masked, if a mixture of phototrophically produced Chlorella vulgaris and heterotrophically produced Chlorella vulgaris was used.

Consequently, the algae may be selected from the group consisting of phototrophically produced algae, heterotrophically fermented algae, or combinations thereof.

Hence, the Chlorella vulgaris algae may be provided as a mixture of phototrophically produced Chlorella vulgaris and heterotrophically produced Chlorella vulgaris.

The photographic production of Chlorella vulgaris uses light as energy source.

The heterotrophic production of Chlorella vulgaris uses another energy source for Chlorella vulgaris, typically organic compounds.

The phototrophical and the heterotrophic production of Chlorella vulgaris is known in the art and for example, well described in Scientific Reports (2019) 9:13935, herein incorporated by reference.

In accordance with the present invention, the algae containing composition may be cooled down between the primary and the secondary heat treatment. For example, after the primary heat treatment the composition may be cooled to a temperature in the range of about 70° C. to 80° C. This has the advantage, for example, that after the primary heat treatment of the algae containing composition, further ingredients may be added. This can be done easier, if the temperature of the composition is below 100° C., for example, if the temperature is between 70° C. and 80° C. This would allow that in the primary heat treatment only algae in water are heat treated, and all further ingredients of the final algae containing composition are only heat treated once. This would allow to better preserve the micronutrients and macronutrients present in the other ingredients present in the final composition. For example, if the final composition is a food composition, micro- and macronutrients can be treated as gently as possible while ensuring food safety.

For example, the primary heat treatment may be carried out by a direct heat treatment, for example on a VTIS (Vacu⋅Therm Instant Sterilizer) line. Such a VTIS line is well known and commercially available, for example under the trademark Tetra Therm® Aseptic VTIS″.

The primary heating step may be carried out by steam injection. Steam injection heating is a technique well known in the art and is, for example, described in Journal of Dairy Science, Volume 43, Issue 11, November 1960, Pages 1693-1696. It is a specific method that can be used during food processing. Steam is directly mixed with the process fluid that needs to be heated. This type of heating has the advantage that it is very efficient and cost-effective compared to other heating methods that often involve contact to heat exchangers.

Hence, during the primary heat treatment hot steam maybe injected into the algae containing composition. The inventors have obtained very good results when the primary heat treatment involved the injection of steam at a temperature in the range of about 130° C. to 160° C. With this method a significant reduction of unwanted algae off-flavors could be achieved.

The primary heat treatment may further involve a pre-heating step, a heating step, a flash-cooling step and/or a downstream homogenization step.

The pre-heating step allows it to prevent that the algae containing composition is subjected to a heat-shock by raising the temperature from room temperature abruptly to 130° C. to 160° C. Hence, in the pre-heating step the temperature of the algae containing composition may be raised to about 70-80° C.

The heating step may be carried out through direct steam injection to about 130° C. to 160° C. for a holding time of about 2 to 10 seconds.

The flash-cooling step may be used to cool the algae containing composition after the heating step to about 70-80° C.

The homogenization step may be carried out at a pressure of about 10-100 bar. Homogenization is typically used to make a mixture of two mutually non-soluble components, for example by producing extremely small particles of one component distributed uniformly throughout the other component.

In particular, if the production of the final composition cannot immediately proceed, and if there is a longer interruption between primary and secondary heat treatment, it may be preferred to cool down the composition after the primary heat treatment to about 0-10° C. To preserve product characteristics as far as possible, it may be preferable if such a cooling step is also carried out by flash cooling.

Hence, in one embodiment of the present invention, the primary heat treatment involves a pre-heating step to about 70-80° C., a heating step through direct steam injection to about 130° C. to 160° C. for a holding time of about 2 to 10 seconds, a flash cooling step to about 70-80° C., and a downstream homogenization step at a pressure of about 10-100 bar. The primary heat treatment may further involve a cooling step to about 0-10° C.

The secondary heat treatment may be carried out on by indirect heat treatment. The indirect heat treatment may be carried out, for example through a tubular heat exchanger or a plate heat exchanger. For example, such a secondary heat treatment may be carried out on a Tetra Flex line. Such Flex line is well known and, for example described in Journal of Food Science, Vol. 76, Nr. 5, 2011, C714-C723.

The secondary heat treatment step may involve a pre-heating step, a heating step, a cooling step, a downstream homogenization step, and/or a cooling step.

The pre-heating step allows it to prevent that the algae containing composition is subjected to a heat-shock by raising the temperature from room temperature abruptly to 100° C. to 150° C. Hence, in the pre-heating step the temperature of the algae containing composition may be raised to about 80° C.-110° C.

The heating step may be carried out at about 100° C. to 150° C. for a holding time of about 2 to 10 seconds.

A cooling step may be used to cool the algae containing composition after the heating step to about 60-80° C.

The downstream homogenization step may be carried out at a pressure of about 200-400 bar. Homogenization is used to make a mixture of mutually non-soluble components, for example by producing extremely small particles of non-soluble components distributed uniformly throughout another component.

Finally, the algae containing composition may be cooled to 20-30° C.

Hence, the secondary heat treatment may involve a pre-heating step to about 80-110° C., a heating step to about 100° C. to 150° C. for a holding time of about 2 to 10 seconds, a cooling step to about 60-80° C., a downstream homogenization step at a pressure of about 200-400 bar, and a cooling step to about 20-30° C.

The method described in the framework of the present invention has the advantage that it allows adding further ingredients after the completion of the primary heating step. This way, not all ingredients have to be subjected to two heating steps, which allows avoiding unnecessary stress for ingredients which do not need to be heat treated twice. Hence, if other desired aroma carriers such as fruit or flavors, for example, are present in the final composition, they can be added to the algae containing composition after the primary heat treatment, so that the desired aromas can be protected and maintained.

Hence, in the method in accordance with the present invention a mixture of algae in water may be subjected to the primary heat treatment. Further ingredients may be added to the composition after the primary heat treatment and the completed composition may then be subjected to the secondary heat treatment.

For example, in the method in accordance with the present invention, the algae containing composition that is subjected to the primary heat treatment may contain and/or consist of algae and water. For example, the algae containing composition that is subjected to the primary heat treatment may contain algae in an amount in the range of about 1-+30% w/w, about 2-25% w/w or about 4-20% w/w. Further for example, the algae containing composition that is subjected to the primary heat treatment may contain algae in an amount in the range of about 4-6% w/w, for example about 5% w/w. This has the advantage, that this algae containing composition can then be combined with all other ingredients of the final composition before the secondary heat treatment in a ratio of about 1:4, so that an algae concentration of 1% w/w with no unwanted off-flavors is obtained in the final composition.

Subjecting a concentrated algae solution to the primary heat treatment has the further advantage that a relatively small volume of an algae containing composition is subjected to the primary heat treatment, which results in energy savings.

For example, the present inventors have prepared several food compositions by subjecting a mixture of algae and water to the primary heat treatment. After completion of the primary heat treatment, all further ingredients were added, and the resulting composition was subjected to the secondary heat treatment, resulting in compositions that were shelf-stable at ambient temperatures and comprised relatively high concentrations of algae with no unpleasant off tastes.

For example, if the algae containing composition is a food composition, further for example a liquid food composition, the composition may contain further ingredients to improve, for example, the nutritional profile, mouthfeel and/or taste of the composition. As such, the liquid food composition may further comprise one or more ingredients selected from the group consisting of sunflower oil, soy protein, citrus pectin, inulin, or combinations thereof.

Even further, the liquid food composition may further comprise a stabilizer. The stabilizer may be gellan gum, for example. As a food additive, gellan gum may be used to bind, stabilize, or texturize food. While it is similar to other gelling agents, it might be preferable because of its effectiveness in low amounts, its clarity and its heat stability. Also, it may be used as a plant-based alternative to gelatin.

If the algae containing composition is a food composition, it was found that the method of the present invention allows it that the food composition has a relatively high algae, for example, Chlorella vulgaris, content. Other drinkable food compositions containing Chlorella vulgaris that are on the market today may contain, for example, between 0.1 and 0.2 weight-% Chlorella vulgaris. In contrast, the method of the present invention allows it to manufacture drinkable compositions without unpleasant algae-off taste comprising at least about 0.3 weight-%, 0.4 weight-%, or 0.5 weight-% Chlorella vulgaris. For example, the liquid food composition may comprise about 0.8-1.2 weight-% Chlorella vulgaris algae.

One of the advantages of Chlorella vulgaris is that it is naturally rich in iron and vitamin B12. Curr Pharm Des. 2016; 22(2):164-73 describes Chlorella vulgaris as a multifunctional dietary supplement with diverse medicinal properties. Chlorella is well known to provide proteins—and with it to contains all nine essential amino acids, vitamin B12, iron, vitamin C, antioxidants, magnesium, zinc, copper, potassium, calcium, folic acid, B vitamins, omega-3 fatty acids and fiber.

As a consequence, the liquid food composition that the method of the present invention allows to prepare may be naturally rich in vitamin B12, iron, proteins and fibers.

FIGS. 1-3 show recipes off typical liquid food compositions that the method of the present invention allows to prepare.

Consequently, the method of the present invention allows it that after the heat treatment the algae containing composition comprises less off flavor caused by algae while maintaining the desired flavor of the remaining components of the composition.

The inventor have further analyzed the algae containing composition before and after subjecting it to the heat treatment described in the framework of the present invention.

The inventors were surprised to see that the heat treatment allowed to significantly reduce several compounds that are naturally present in algae containing compositions and that cause unpleasant off taste.

Hence, in the method of the present invention after the heat treatment the algae containing composition comprises less off flavor caused by one or more compounds selected from the group consisting of 2-methylbutanal, 3-methylbutanal, 1-ethenyl-aziridine, 3-hexanone, hexanal, 2-methyl-2-butenal, 2-methyl-2-pentenal, 1-penten-3-ol, 2-heptanone, heptanal, octanal, 6-methyl-5-hepten-2-one, 3E,5Z-octadien-2-one, 3E,5E-octadien-2-one, benzaldehyde, acetophenone, 2-methylpentanoic acid, or combinations thereof.

The subject matter of the present invention includes compositions obtained by and/or obtainable with the method of the present invention.

Hence, the subject matter of the present invention includes an algae containing composition comprising a reduced content of one or more compounds selected from the group consisting of 2-methylbutanal, 3-methylbutanal, 1-ethenyl-aziridine, 3-hexanone, hexanal, 2-methyl-2-butenal, 2-methyl-2-pentenal, 1-penten-3-ol, 2-heptanone, heptanal, octanal, 6-methyl-5-hepten-2-one, 3E,5Z-octadien-2-one, 3E,5E-octadien-2-one, benzaldehyde, acetophenone, 2-methylpentanoic acid, or combinations thereof, compared to the mixture of the algae in water.

For example, the content of these compounds may be reduced by at least 50%, at least 60%, at least 70%, at least 80% or at least 90%.

### Food Composition Comprising Apple, Guava and Algae

The invention further relates to a liquid food composition comprising water, apple puree, guava puree, and Chlorella vulgaris algae, wherein the Chlorella vulgaris algae are provided as a mixture of phototrophically produced Chlorella vulgaris and heterotrophically produced Chlorella vulgaris.

In one embodiment, the phototrophically produced Chlorella vulgaris and the heterotrophically produced Chlorella vulgaris are provided in a weight ratio of phototrophically produced Chlorella vulgaris to heterotrophically produced Chlorella vulgaris in the range of about 1:1 to 3:1, about 1.5:1 to 2.5:1, or about 1.8:1 to 2.2:1. Very good results were obtained when the weight ratio phototrophically produced Chlorella vulgaris to heterotrophically produced Chlorella vulgaris was in the range of about 2:1.

In one embodiment, the liquid food composition further comprises one or more ingredients selected from the group consisting of sunflower oil, soy protein, citrus pectin, inulin, or combinations thereof.

In one embodiment, the liquid food composition further comprises a stabilizer, for example gellan gum.

In one embodiment, the composition comprises about 10-14 weight-% apple puree, about 6-10 weight-% guava puree and/or about 0.8-1.2 weight-% Chlorella vulgaris algae.

In one embodiment, the composition comprises about 0.8-1.2 weight-% sunflower oil, about 0.1-1.1 weight-% soy protein, about 0.2-0.7 weight-% citrus pectin, and/or about 0.4-0.8 weight-% inulin. Sunflower oil has the advantage that it is easily available and that it is primarily composed of linoleic acid, a polyunsaturated fat, and oleic acid, a monounsaturated fat. High oleic sunflower oil may be used.

In one embodiment, the composition further comprises about 8-12 weight-% oat syrup, and/or about 0.3-0.7 weight-% oat flower.

In one embodiment, the composition further comprises one or more flavoring agents.

In one embodiment, the food composition has an energy density in the range of about 50-70 kcal/100 ml, for example in the range of about 55-65 kcal/100 ml.

In one embodiment, the food composition comprises per 100 ml about 1.8-2.2 g protein, about 1.5-1.7 g fat, about 7.0-9.5 g carbohydrates, and about 1.8-2.2 g fiber.

In one embodiment, the liquid food composition further comprises vitamins and minerals selected from the group consisting of iron and vitamin B12. For example, the liquid food composition may comprise per 100 ml about 0.8-1.2 mg iron, and/or about 0.3-1.0 μg vitamin B12.

In one embodiment, the liquid food composition comprises per 100 ml about 5.0-7.0 g sugar.

In one embodiment, the composition may comprise about 60-65 weight-% water, about 10-12 weight-% apple puree, about 10-12 weight-% oat syrup, about 7-9 weight-% guava puree, about 0.8-1.0 weight-% soy protein isolate, about 0.9-1.1 weight-% Chlorella vulgaris, about 0.9-1.1 weight-% sunflower oil, about 0.5-0.7 weight-% inulin, about 0.4-0.6 weight-% lemon juice concentrate, about 0.2-0.3 weight-% citrus pectin, and about 0.01-0.02 weight-% gellan gum.

In one embodiment, the algae and/or the composition are heat treated.

In one embodiment, the liquid food composition is a vegetarian or vegan composition.

The flavor attributes of two different types of Chlorella were tested. Two samples were prepared by mixing a total concentration of Chlorella algae with water and evaluated sensorially by a panel of 5 people. The sensory properties of a mix of phototrophic (0.65%) and heterotrophic (0.35%) Chlorella was rated significantly better compared to the same amount of only phototrophic Chlorella (1%). Especially undesired algae taste, odor and aftertaste is reduced.

In total, 8 samples were evaluated in a blind tasting by 16 tasters in order to test the impact of the selected attributes on the intensity of the algae taste perception. The samples all contained the same amount and same type of algae. The plant-based matrix (here oat-base) showed a better algae masking potential compared to a dairy matrix. The JAR score of the attribute Matrix was +0.6 for Dairy (algae taste was considered as too strong) compared to a score of −0.2 for plant-based. The taste direction (fruity vs non-fruity) was the attribute with the second highest impact on the algae taste perception. Also, with increased sweetness the perception of algae is reduced.

Based on the data obtained, the principle recipe structure (oat-based with fruits having a certain sweetness) and the final recipe was developed, and masking potential was confirmed as predicted based on trials described above.

### Food Composition Comprising per Puree, Cocoa, and Algae

The invention further relates to a liquid food composition comprising water, pear puree, cocoa, and Chlorella vulgaris algae, wherein the Chlorella vulgaris algae are provided as a mixture of phototrophically produced Chlorella vulgaris and heterotrophically produced Chlorella vulgaris.

In one embodiment, the phototrophically produced Chlorella vulgaris and the heterotrophically produced Chlorella vulgaris are provided in a weight ratio of phototrophically produced Chlorella vulgaris to heterotrophically produced Chlorella vulgaris in the range of about 1:1 to 3:1, about 1.5:1 to 2.5:1, or about 1.8:1 to 2.2:1. Very good results were obtained when the weight ratio phototrophically produced Chlorella vulgaris to heterotrophically produced Chlorella vulgaris was in the range of about 2:1.

In one embodiment, the liquid food composition further comprises one or more ingredients selected from the group consisting of sunflower oil, soy protein, citrus pectin, inulin, or combinations thereof.

In one embodiment, the liquid food composition further comprises a stabilizer, for example gellan gum.

In one embodiment, the composition comprises about 17-21 weight-% pear puree, about 0.8 —1.2 weight-% cocoa and/or about 0.8-1.2 weight-% Chlorella vulgaris algae.

In one embodiment, the composition comprises about 0.8-1.2 weight-% sunflower oil, about 0.6-1.0 weight-% soy protein, about 0.2-0.7 weight-% citrus pectin, and/or about 0.1-0.5 weight-% inulin. Sunflower oil has the advantage that it is easily available and that it is primarily composed of linoleic acid, a polyunsaturated fat, and oleic acid, a monounsaturated fat. High oleic sunflower oil may be used.

In one embodiment, the composition further comprises about 10-15 weight-% oat syrup, and/or about 0.3-0.7 weight-% oat flower.

In one embodiment, the composition further comprises one or more flavoring agents.

In one embodiment, the food composition has an energy density in the range of about 60-80 kcal/100 ml, for example in the range of about 65-75 kcal/100 ml.

In one embodiment, the food composition comprises per 100 ml about 2.0-2.4 g protein, about 1.8-2.2 g fat, about 8.0-11.0 g carbohydrates, and about 1.8-2.3 g fiber.

In one embodiment, the liquid food composition further comprises vitamins and minerals selected from the group consisting of iron and vitamin B12. For example, the liquid food composition may comprise per 100 ml about 0.8-1.2 mg iron, and/or about 0.3-1.0 μg vitamin B12.

In one embodiment, the liquid food composition comprises per 100 ml about 5.0-7.0 g sugar.

In one embodiment, the composition may comprise about 60-65 weight-% water, about 15-20 weight-% pear puree, about 12-15 weight-% oat syrup, about 0.8-1.2 weight-% cocoa, about 0.8-1.0 weight-% soy protein isolate, about 0.9-1.1 weight-% Chlorella vulgaris, about 0.9-1.1 weight-% sunflower oil, about 0.2-0.5 weight-% inulin, about 0.5-1.0 weight-% lemon juice concentrate, about 0.2-0.3 weight-% citrus pectin, and about 0.01-0.02 weight-% gellan gum.

In one embodiment, the algae and/or the composition are heat treated.

In one embodiment, the liquid food composition is a vegetarian or vegan composition.

The flavor attributes of two different types of Chlorella were tested. Two samples were prepared by mixing a total concentration of Chlorella algae with water and evaluated sensorially by a panel of 5 people. The sensory properties of a mix of phototrophic (0.65%) and heterotrophic (0.35%) Chlorella was rated significantly better compared to the same amount of only phototrophic Chlorella (1%). Especially undesired algae taste, odor and aftertaste is reduced.

In total, 8 samples were evaluated in a blind tasting by 16 tasters in order to test the impact of the selected attributes on the intensity of the algae taste perception. The samples all contained the same amount and same type of algae. The plant-based matrix (here oat-base) showed a better algae masking potential compared to a dairy matrix. The JAR score of the attribute Matrix was +0.6 for Dairy (algae taste was considered as too strong) compared to a score of −0.2 for plant-based. The taste direction (fruity vs non-fruity) was the attribute with the second highest impact on the algae taste perception. Also, with increased sweetness the perception of algae is reduced.

Based on the data obtained, the principle recipe structure (oat-based with fruits having a certain sweetness) and the final recipe was developed, and masking potential was confirmed as predicted based on trials described above.

### Food Composition Comprising Mango, Lemongrass Flavour, and Algae

The invention further relates to a liquid food composition comprising water, mango puree, lemongrass flavour, and Chlorella vulgaris algae, wherein the Chlorella vulgaris algae are provided as a mixture of phototrophically produced Chlorella vulgaris and heterotrophically produced Chlorella vulgaris.

In one embodiment, the phototrophically produced Chlorella vulgaris and the heterotrophically produced Chlorella vulgaris are provided in a weight ratio of phototrophically produced Chlorella vulgaris to heterotrophically produced Chlorella vulgaris in the range of about 1:1 to 3:1, about 1.5:1 to 2.5:1, or about 1.8:1 to 2.2:1. Very good results were obtained when the weight ratio phototrophically produced Chlorella vulgaris to heterotrophically produced Chlorella vulgaris was in the range of about 2:1.

In one embodiment, the liquid food composition further comprises one or more ingredients selected from the group consisting of sunflower oil, soy protein, citrus pectin, inulin, or combinations thereof.

In one embodiment, the liquid food composition further comprises a stabilizer, for example gellan gum.

In one embodiment, the composition comprises about 15-22 weight-% mango puree, and/or about 0.8-1.5 weight-% Chlorella vulgaris algae.

In one embodiment, the composition comprises about 0.8-1.2 weight-% sunflower oil, about 0.9-1.3 weight-% soy protein, about 0.2-0.7 weight-% citrus pectin, and/or about 0.6-1.0 weight-% inulin. Sunflower oil has the advantage that it is easily available and that it is primarily composed of linoleic acid, a polyunsaturated fat, and oleic acid, a monounsaturated fat. High oleic sunflower oil may be used.

In one embodiment, the composition further comprises about 9-13 weight-% oat syrup, and/or about 0.3-0.7 weight-% oat flower.

In one embodiment, the composition further comprises one or more flavoring agents.

In one embodiment, the food composition has an energy density in the range of about 60-80 kcal/100 ml, for example in the range of about 65-75 kcal/100 ml.

In one embodiment, the food composition comprises per 100 ml about 1.9-2.3 g protein, about 1.6-1.8 g fat, about 8.0-10.5 g carbohydrates, and about 1.8-2.2 g fiber.

In one embodiment, the liquid food composition further comprises vitamins and minerals selected from the group consisting of iron and vitamin B12. For example, the liquid food composition may comprise per 100 ml about 0.8-1.2 mg iron, and/or about 0.3-1.0 μg vitamin B12.

In one embodiment, the liquid food composition comprises per 100 ml about 6.0-8.0 g sugar.

In one embodiment, the composition may comprise about 60-65 weight-% water, about 19-21 weight-% mango puree, about 12-13 weight-% oat syrup, about 1.0-1.1 weight-% soy protein isolate, about 0.9-1.1 weight-% Chlorella vulgaris, about 0.9-1.1 weight-% sunflower oil, about 0.7-0.9 weight-% inulin, about 0.4-0.5 weight-% lemon juice concentrate, about 0.2-0.3 weight-% citrus pectin, and about 0.01-0.02 weight-% gellan gum.

In one embodiment, the algae and/or the composition are heat treated.

In one embodiment, the liquid food composition is a vegetarian or vegan composition.

The flavor attributes of two different types of Chlorella were tested. Two samples were prepared by mixing a total concentration of Chlorella algae with water and evaluated sensorially by a panel of 5 people. The sensory properties of a mix of phototrophic (0.65%) and heterotrophic (0.35%) Chlorella was rated significantly better compared to the same amount of only phototrophic Chlorella (1%). Especially undesired algae taste, odor and aftertaste is reduced.

In total, 8 samples were evaluated in a blind tasting by 16 tasters in order to test the impact of the selected attributes on the intensity of the algae taste perception. The samples all contained the same amount and same type of algae. The plant-based matrix (here oat-base) showed a better algae masking potential compared to a dairy matrix. The JAR score of the attribute Matrix was +0.6 for Dairy (algae taste was considered as too strong) compared to a score of −0.2 for plant-based. The taste direction (fruity vs non-fruity) was the attribute with the second highest impact on the algae taste perception. Also, with increased sweetness the perception of algae is reduced.

Based on the data obtained, the principle recipe structure (oat-based with fruits having a certain sweetness) and the final recipe was developed, and masking potential was confirmed as predicted based on trials described above.

Those skilled in the art will understand that they can freely combine all features of the present invention disclosed herein. In particular, features described for the method of the present invention may be combined with the composition of the present invention and vice versa. Further, features described for different embodiments of the present invention may be combined.

Although the invention has been described by way of example, it should be appreciated that variations and modifications may be made without departing from the scope of the invention as defined in the claims.

Furthermore, where known equivalents exist to specific features, such equivalents are incorporated as if specifically referred in this specification. Further advantages and features of the present invention are apparent from the figures and non-limiting examples.

### EXAMPLES

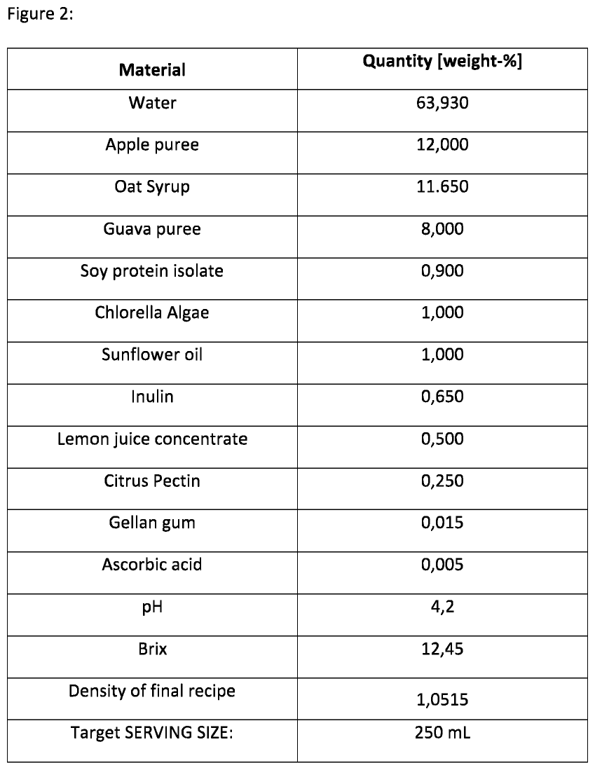

The impact of three different processes on the sensory attributes of a drink containing Chlorella algae was evaluated. The same recipe, described in FIG. 2, was used for all three runs with the following process variations:

Sample 1 (conventional indirect heat treatment): Mixing of all ingredients—indirect heat treatment—homogenization

Sample 2 (conventional direct heat treatment): Mixing of all ingredients—direct heat treatment—homogenization

Sample 3 (novel 2-step process described above): Preparation of algae stock solution—direct heat treatment of the algae solution—mixing of algae solution with all other ingredients—indirect heat treatment of the mix—homogenization

The three samples were evaluated sensorially by a panel of 5 people. As shown in FIG. 4, the sensory properties of sample 3 (novel 2-step process) was rated significantly better compared to the other two conventional approaches. The undesired algae typical flavor and odor was significantly reduced while the desired fruit flavor was obtained.

The reduction of the undesired algae typical aroma compounds was, furthermore, analyzed by GC-MS and respective chromatograms are shown in FIG. 5. A Chlorella algae stock solution (5% w/w) was prepared and samples were taken and analyzed before and after direct heat treatment at 145° C., for 5 s followed by a flash cooling to 75° C.

1. Method to reduce the off-taste of an algae containing composition comprising a heat treatment, wherein the heat treatment comprises a primary heat treatment at a temperature in the range of about 130-160° C. and a secondary heat treatment at a temperature in the range of about 90-150° C.

2. Method in accordance with claim 1, wherein after the primary heat treatment the composition is cooled to a temperature in the range of about 70° C. to 80° C.

3. Method in accordance with claim 1, wherein the primary heat treatment is a heating step carried out by direct steam injection.

4. Method in accordance with claim 3, wherein the primary heat treatment involves the injection of steam at a temperature in the range of about 130° C. to 160° C.

5. Method in accordance with claim 3, wherein the primary heat treatment involves a pre-heating step to about 70-80° C., a heating step through direct steam injection to about 130° C. to 160° C. for a holding time of about 2 to 10 seconds, a flash cooling step to about 70-80° C., a downstream homogenization step at a pressure of about 10-100 bar, and a cooling step to about 0-10° C.

6. Method in accordance with claim 1, wherein the primary heat treatment is carried out by a direct heat treatment.

7. Method in accordance with claim 1, wherein after the secondary heat treatment the composition is cooled to a temperature in the range of about 60° C. to 80° C.

8. Method in accordance with claim 1, wherein the secondary heat treatment is a heating step carried out by an indirect heat treatment.

9. Method in accordance with claim 1, wherein the secondary heat treatment involves a pre-heating step to about 80-110° C., a heating step to about 100° C. to 150° C. for a holding time of about 2 to 10 seconds, a cooling step to about 60-80° C., a downstream homogenization step at a pressure of about 200-400 bar, and a cooling step to about 20-30° C.

10. Method in accordance with claim 1, wherein a mixture of algae in water is subjected to the primary heat treatment, further ingredients are added to the composition after the primary heat treatment and the completed composition is then subjected to the secondary heat treatment.

11. Method in accordance with claim 1, wherein the algae selected from the genus Chlorella.

12. Method in accordance with claim 1, wherein after the heat treatment the algae containing composition comprises less off flavor caused by algae while maintaining the desired flavor of the remaining components of the composition.

13. Method in accordance with claim 1, wherein after the heat treatment the algae containing composition comprises less off flavor caused by one or more compounds selected from the group consisting of 2-methylbutanal, 3-methylbutanal, 1-ethenyl-aziridine, 3-hexanone, hexanal, 2-methyl-2-butenal, 2-methyl-2-pentenal, 1-penten-3-ol, 2-heptanone, heptanal, octanal, 6-methyl-5-hepten-2-one, 3E,5Z-octadien-2-one, 3E,5E-octadien-2-one, benzaldehyde, acetophenone, 2-methylpentanoic acid, and combinations thereof.

14. Method in accordance with claim 1, wherein the algae containing composition is a food product.

15. (canceled)
